(19) United States
(12) Patent Application Publication
(10) Pub. No.: US 2026/0031240 A1
(43) Pub. Date: Jan. 29, 2026

(54) SYSTEM AND METHOD FOR CALCULATING VALUE CREATION IN DELEGATED RISK CONTRACTS USING HEALTHCARE CLAIMS DATA

(71) Applicant: Parallel Health, LLC, Franklin, TN (US)

(72) Inventor: Aaron R. Wells, Franklin, TN (US)

(21) Appl. No.: 19/257,577

(22) Filed: Jul. 2, 2025

Related U.S. Application Data
(60) Provisional application No. 63/675,205, filed on Jul. 24, 2024.

(51) Int. Cl. G16H 50/70; G16H 40/20

(52) U.S. Cl. CPC G16H 50/70; G16H 40/20

(57) ABSTRACT

A value estimation method for a delegated healthcare model generating a user interface to receive patient-generated data from multiple sources, transforming this data based on predefined parameters, and training a predictive analytics model with this data. The model generates baseline ratings for risk, utilization, and causal inference. Using these baseline ratings, the method predicts distinct risk ratings, healthcare utilization ratings, and causal impact ratings. A value creation estimate, indicating per member per month cost adjustment and utilization adjustment, is then generated based on these predictions and predefined parameters. The delegated model is reconciled based on comparing the value creation estimate to the observed value for the same period of time.

A portion of the disclosure of this patent document contains material that is subject to copyright protection. The copyright owner has no objection to the reproduction of the patent document or the patent disclosure, as it appears in the U.S. Patent and Trademark Office (USPTO) patent file or records, but otherwise reserves all copyright rights whatsoever.

### FIELD OF THE DISCLOSURE

The present invention relates to the filed of healthcare data analytics, for example methods and systems for predictive modeling and actuarial risk assessment within value-based care (VBC) contracting frameworks.

### BACKGROUND

In recent years, the healthcare industry has increasingly shifted towards VBC models, which focus on patient outcomes rather than the volume of services provided. This shift necessitates advanced techniques for accurately predicting healthcare costs, utilization patterns, and financial risks associated with different patient populations. Effective predictive modeling enables healthcare providers and insurers to optimize resource allocation, manage costs, and improve patient care.

One important aspect of the transformation is the actuarial risk assessment, which involves estimating the financial impact of different risk categories on overall health expenditures. Actuarial methods help in stratifying the insured population into various risk categories based on demographic, clinical, and utilization data. Predictive models are then employed to calculate the expected cost distribution for each risk category and project the total financial risk for the insured population under delegated risk contracts. This process ensures that healthcare organizations can anticipate financial liabilities and manage risks effectively, ensuring sustainability in the VBC landscape.

In developing models, it is essential to utilize high-quality datasets representative of the broader population. The datasets should undergo rigorous preprocessing, including data cleaning, feature engineering, population stratification, bootstrapping, and the application of business rules to truncate, censor, or exclude specific data points. Common parameters such as demographic information, clinical data, healthcare utilization patterns, and insurance details are crucial for training models that can accurately forecast metrics like Medical Loss Ratio (MLR), Total Cost of Care (TCoC), and acute utilization. Ensuring the training dataset mirrors distributional characteristics of the application dataset enhances the model's generalizability and accuracy.

However, these previous methods have several deficiencies. Existing approaches often struggle with handling the complexity and variability or healthcare data, leading to inaccuracies in risk stratification and cost predictions. Many predictive modes fail to adequately address the impact of outliers and extreme values, resulting in skewed analyses. Furthermore, traditional actuarial methods may not fully leverage advanced machine learning techniques or causal inference methods, limiting their ability to accurately estimate the financial impact of interventions or policy changes. The lack of robust preprocessing and data transformation steps can also compromise the quality and reliability of the predictive models.

Nonlimiting advantages of the present disclosure include integrating advanced predictive modeling techniques, comprehensive data preprocessing methods, and rigorous causal inference approaches. The present disclosure ensures that healthcare datasets are thoroughly cleaned, standardized, and transformed, resulting in high-quality inputs for predictive modeling. By employing methods such as Propensity Score Matching (PSM), Difference-in-Difference (DiD) analysis, supervised and unsupervised classification, and Instrumental Variables (IV) techniques, the invention provides more accurate estimates of causal impacts and financial risks. Additionally, the use of sophisticated machine learning algorithms enhances the precision of cost prediction and risk assessments, leading to better resource allocation and improved patient outcomes in a value-based care environment.

### BRIEF SUMMARY

The present disclosure provides a novel value estimation method for a delegated model. Specifically, the present disclosure provides a novel value estimation method, as well as a system for implementing the value estimation method.

Embodiments of apparatus, methods, and systems of the present disclosure provide a solution to the shortcomings about. In particular, this disclosure provides a value estimation method. In some aspects, the techniques described herein related to a value estimation method for a delegated risk model, including: generating, at a server, a user interface configured to receive a first set of patient-generated data from one or more of a plurality of data sources associated with the delegated model, wherein the first set of patient-generated data includes a plurality of covariates; transforming, based at least upon a predefined delegated modem parameter, the first set of patient-generated data into a second set of patient-generated data, wherein the predefined delegated model parameter is variably determined by one or more of a plurality of entities associated with the delegated model; training a predictive analytics model by inputting into the predictive analytics model at least a baseline set of patient-generated data and the predefined delegated model parameter, where the baseline set of patient-generated data is representatively associated with the first set of patient-generated data, the second set of patient-generated data, or both, and where training the predictive analytics model is configured to generated a baseline risk rating, a baseline utilization rating, and a baseline causal inference rating; predicting at least one distinct risk rating, based on the predictive analytics model, the baseline risk rating, the second set of patient-generated data; predicting a healthcare utilization rating, based on predictive analytics model, the baseline utilization rating, and the second set of patient generated data; predicting causal impact rating, based on the predictive analytics model, the baseline causal inference rating, and the seconds set of patient-generated data; generating a value creation estimate in the delegated model based upon the predefined delegated model parameter, the at least one distinct risk rating, the healthcare utilization rating, and the causal impact rating; presenting, by the server, for display on a user device, the value creation estimate in natural language text, wherein the natural language test indicates at least a per member per month cost adjustment and a utilization adjustment; reconciling the delegated model based upon the value creation estimate.

In some aspects, the techniques described herein related to a value estimation method, wherein the step of transforming includes: applying the predefined delegated model parameter to the first set of patient-generated data according to a user-defined time period; enhancing the first set of patient-generated data with an engineered feature according to the user-defined time period, or a combination thereof; applying one or more standardization rules to the first set of patient-generated date; normalizing the first set of patient-generated data at a member level.

In some aspects, the techniques described herein related to a value estimation method, wherein the training includes: stratifying the baseline set of patient-generated data into a plurality of distinct risk categories based upon the predictive analytics model; assigning a category risk rating to each distinct risk category of the plurality of distinct risk categories; generating an expected cost distribution for each distinct risk category of the plurality of distinct risk categories.

In some aspects, the techniques described herein relate to a value estimation method, wherein the training includes: receiving, via user-generated input, a baseline risk scenario reflecting one or more user-defined assumptions, wherein the user-defined assumption include healthcare costs, demographic shifts, utilization patterns, or policy changes; generating, by the server, one or more alternative risk scenarios, wherein each of the one or more alternative risk scenarios alters at least one of the one or more user-defining assumptions; comparing, by the server and via the predictive analytics model, each of the one or more alternative risk scenarios to the baseline risk scenario; generating a sensitivity rating for each of the comparisons of each of the one or more alternative risk scenarios to the baseline risk scenario; adjusting the baseline risk rating based upon the sensitivity rating.

In some aspects, the techniques described herein relate to a value estimation method, wherein the training includes: defining, from the baseline set of patient-generated data, a treatment group and a control group; selecting a treatment subset of the plurality of covariates, wherein the treatment subset indicates a method of treatment; estimating a multivariate propensity score of the baseline set of patient-generated data based at least upon the treatment; matching the treatment group and the control group based on the propensity score.

In some aspects, the techniques described herein relate to a value estimation method, where: each covariate may provide health benefits eligibility and utilization, demographics, geospatial data, and combinations thereof.

Unless otherwise defined, all technical and scientific terms used herein have the same meaning as commonly understood by one of ordinary skill in the art to which this invention belongs. The present invention may be embodied in other specific forms without departing from the spirit or essential attributes thereof, and it is therefore desired that the present embodiment be considered in all aspects as illustrative and not restrictive. Any headings utilized in the description are for convenience only and no legal or limiting effect. Numerous objects, features, and advantages of the embodiments set forth herein will be readily apparent to those skilled in the art upon reading of the following disclosure when taken in conjunction with the accompanying drawings.

### BRIEF DESCRIPTION OF THE DRAWINGS

Hereinafter, various exemplary embodiments of the disclosure are illustrated in more detail with reference to the drawings.

FIGS. 1A and 1B are a flowchart representing an embodiment of a value estimation method according to the present disclosure.

FIG. 2 is a block diagram representing an embodiment of a value estimation system according to the present disclosure.

FIG. 3 is a block diagram representing an embodiment of a processor according to the value estimation system.

### DETAILED DESCRIPTION

Reference will now be made in detail to embodiments of the present disclosure, one or more drawings of which are set forth herein. Each drawing is provided by way of explanation of the present disclosure and is not a limitation. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

Thus, it is intended that the present disclosure covers such modifications and variations as come within the scope of the appended claims and their equivalents. Other objects, features, and aspects of the present disclosure are disclosed in, or are obvious from, the following detailed description. It is to be understood by one of ordinary skill in the art that the present discussion is a description of exemplary aspects of the disclosure only and is not intended as limiting the broader aspects of the present disclosure.

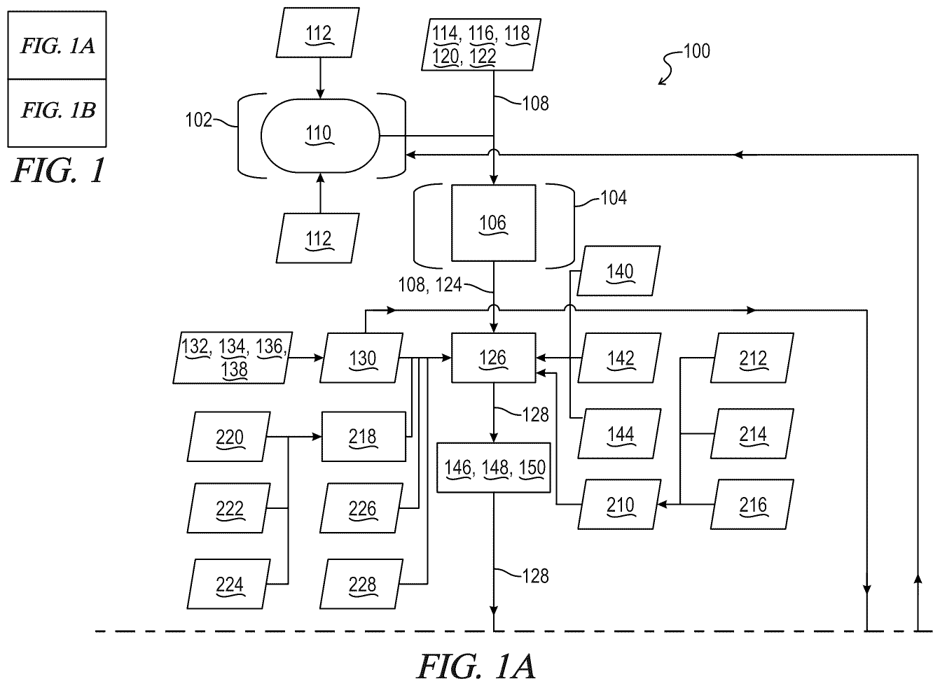

Referring generally to FIGS. 1A-3, various exemplary aspects may now be described of a value estimation method 100 and systems of implementation thereof. Specifically, various aspects may now be described of the value estimation method 100 and a value estimation system 300 for executing the value estimation method 100. Where the various figures describe aspects sharing various common elements and features with other aspects, similar elements and features are given the same reference numerals and redundant description thereof may be omitted below.

Various embodiments of an invention may be described below with reference to block diagrams and flowchart illustrations of methods, apparatus (i.e., systems) and computer program products (i.e., computer executable instruction modules). It will be understood by one of skill in the art that each block of the block diagrams and the flowchart illustrations, and combinations of blocks in the block diagrams and combinations of the blocks in the flowchart illustrations, can be implemented by computer program instructions. These computer program instructions may be loaded onto a general purpose computer, special purpose computer, or other programmable data processing apparatus to produce a machine, such that the instructions that execute on the computer or other programmable data processing apparatus create means for implementing the functions specified in the block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function specified in the block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the block or blocks of the flowchart, or block or blocks of the diagrams.

Accordingly, blocks of the block diagrams and the flowchart illustrations support combinations of means for performing the specified functions, combinations of steps for performing the specified functions and program instructions or modules for performing the specified functions. It will also be understood that each block of the block diagrams and the flowchart illustrations, and combinations of the respective blocks, can be implemented by special purpose hardware-based computer systems which perform the specified functions or steps, or combinations of special purpose hardware and computer instructions.

FIGS. 1A and 1B illustrate an aspect of the value estimation method 100 of the present disclosure. The value estimation method 100 may be employed within a delegated model 102. The delegated model 102 may represent a delegated risk model or a delegated risk contract in which an entity assumes financial responsibility for providing or arranging for the healthcare services needed by a defined population of patients.

The value estimation method incorporates several processes to accurately predict and assess healthcare values within a delegated model framework. It begins with enhanced data preprocessing, involving the receipt of patient-generated data from multiple sources and applying sophisticated data cleaning, normalization, and transformation techniques to ensure consistency and reliability. The value estimation method 100 also provides enhanced risk stratification, where the preprocessed data is analyzed to classify patients into different risk categories based on various covariates such as demographics, clinical history, and utilization patterns. Advanced statistical and machine learning techniques accurately capture the nuanced risk profiles of the patient population. Enhanced risk prediction then employs a predictive analytics model trained using the stratified data and predefined parameters, generating baseline ratings for risk, utilization, and causal inference, and predicting distinct risk ratings, healthcare utilization ratings, and causal impact ratings for individuals or groups. Finally, the value estimation method 100 provides enhanced reconciliation of the observed outcome variable by comparing observed outcomes, such as Total Cost of Care (TCoC), with expected values retrospectively over equivalent periods. This reconciliation adjusts for discrepancies between predicted and actual values, validating the predictive model's accuracy and refining it for better future performance. The value estimation method 100 provides a robust framework for predicting healthcare values, managing financial risks, and improving patient outcomes in a value-based care environment.

The value estimation method 100 may include a step of generating 104 a user interface 106 that is configured to receive a first set of patient-generated data 108 from one or more of a plurality of data sources 110 associated with the delegated model 102. Each of the plurality of data sources 110 associated with the delegated model 102 may be an entity 112 associated with the delegated model 102, including, but not limited to, a party to an Alternative Payment Model (APM) contract.

The first set of patient-generated data 108 may include at least a plurality of covariates, including qualitative health assessment data, medical and pharmacy claims, health benefits eligibility, demographics, geospatial data, and combinations thereof. It will be understood by those of ordinary skill in the art that the plurality of covariates may include any variable or parameter relevant to the value estimation method 100 as collected from the first set of patient-generated data 108.

The first set of patient-generated data 108 may include information related to various aspects of patient information relevant to assessing healthcare usage, quality of healthcare provided, and associated costs. The first set of patient-generated data 108 may include qualitative health assessment data 114, medical and pharmacy claims data 116, health benefits eligibility data 118, demographic information 120, and geospatial data 122.

Qualitative health assessment data 114 may encompass a wide range of subjective information that provides insights into a patient's health status and experiences. This type of data may include patient-reported outcomes, health surveys, and clinical assessments that capture the nuances of a patient's health beyond quantitative metrics. Examples of qualitative health assessment data 114 may include the following: (1) Patient Satisfaction Surveys: Surveys that gather feedback on a patient's experience with healthcare services, including their interactions with healthcare providers and overall satisfaction with care; (2) Clinical Notes: Detailed notes written by healthcare providers during consultations that may include observations about a patient's condition, lifestyle factors, and other relevant qualitative information; (3) Psychosocial Assessments: Evaluations that explore a patient's mental health, emotional well-being, and social circumstances, which may impact their overall health; and (4) Patient Interviews: Recorded or transcribed interviews where patients discuss their symptoms, treatment experiences, and health concerns in their own words.

Medical and pharmacy claims data 116 may include comprehensive information about the healthcare services and medications that patients receive. This data is typically generated through billing processes and may provide detailed insights into the cost, frequency, and types of healthcare utilization. Examples of medical and pharmacy claims data 116 may include the following: (1) Medical Claims: Records of healthcare services billed to insurance providers, which may include information such as the date of service, type of service, provider details, diagnosis codes (e.g., ICD-10), and procedure codes (e.g., CPT); (2) Pharmacy Claims: Data on prescribed medications, including the drug name, dosage, quantity, prescribing physician, and pharmacy details; (3) Claims Status: Information on the status of claims, such as whether they are pending, approved, denied, or adjusted; and (4) Cost Information: Details on the billed amount, paid amount, member cost-sharing amounts (e.g., co-pays, deductibles), and any adjustments or discounts applied.

Health benefits eligibility data 118 may include information that determines a patient's eligibility for various health insurance benefits and services. This data may ensure that patients receive the appropriate coverage and care. Examples of health benefits eligibility data 118 may include the following: (1) Enrollment Information: Data on the patient's enrollment in a health insurance plan, including the start and end dates of coverage; (2) Benefit Plan Details: Specifics about the health insurance plan, such as covered services, exclusions, limits, and co-payment structures; (3) Eligibility Verification: Records verifying a patient's eligibility for specific services or benefits at the time of service; and (4) Dependent Coverage: Information about covered dependents, including their eligibility and relationship to the primary insured member.

Demographic information 120 may encompass various personal details about patients that are relevant for healthcare analysis and service delivery. This data may help in understanding population health trends, tailoring interventions, and ensuring equitable access to care. Examples of demographic information 120 may include the following: (1) Age: The patient's age, which may influence their risk factors and healthcare needs; (2) Gender: The patient's gender, which may impact their health risks and treatment options; (3) Race and Ethnicity: Information on the patient's racial and ethnic background, which may be relevant for identifying health disparities and tailoring culturally appropriate care; (4) Marital Status: The patient's marital status, which may affect their social support systems and health behaviors; (5) Income Level: Information about the patient's income, which may be used to assess their socioeconomic status and access to healthcare resources; and (6) Education Level: The patient's educational attainment, which may influence their health literacy and engagement in healthcare.

Geospatial data 122 may include location-based information that provides insights into the geographic distribution of health outcomes, healthcare services, and environmental factors affecting health. This data may be used for public health planning, resource allocation, and identifying geographic health disparities. Examples of geospatial data 122 may include the following: (1) Patient Address: The residential address of patients, which may be used to analyze access to healthcare services and environmental health risks; (2) Healthcare Provider Locations: Information on the locations of healthcare facilities, pharmacies, and providers, which may be used to assess service availability and accessibility; (3) Population Density: Data on the population density in different geographic areas, which may help in understanding the demand for healthcare services; (4) Environmental Data: Information on environmental factors, such as air quality, pollution levels, and proximity to green spaces, which may impact public health; and (5) Transportation Data: Information on transportation networks and accessibility, which may affect patients' ability to reach healthcare services.

The step of generating 104 the user interface 106 may include generating a plurality of user interfaces 106 to allow for each of the plurality of data sources 110 and/or a plurality of entities 112 to provide the first set of patient-generated data 108. In an exemplary aspect, the first set of patient-generated data 108 provided from one of the plurality of data sources 110 may further include a first set of associated metadata 124. The first set of associated metadata 124 may be individually associated with each of the plurality of data sources 110 and may provide additional information about the plurality of data sources 110, the entity 112, or a combination thereof. Without limitation, the first set of associated metadata 124 may include demographic metadata, utilization metadata, financial metadata, performance and quality metrics, clinical and health outcomes metadata, temporal metadata, compliance and regulatory metadata.

Some examples of demographic metadata may include a unique identifier assigned to each insured member, often referred to as the Member ID. Demographic metadata may also encompass essential personal details such as the member's age, gender, and geographic location, which may indicate where the member resides. Additionally, it may record the enrollment date, marking the start of the member's coverage under the healthcare plan, and may identify the type of plan the member is enrolled in, such as HMO or PPO.

Some examples of utilization metadata may include detailed information about the healthcare services received by the member. Utilization metadata may include the service date, which specifies when the healthcare service was provided, and the service type, which may categorize the nature of the service, such as outpatient, inpatient, or emergency care. It may also include identifiers for the healthcare provider (Provider ID) and the facility (Facility ID) where the service was delivered. Furthermore, utilization metadata may encompass procedure codes and diagnosis codes, which describe the specific procedures performed and the diagnoses made, respectively, along with the service quantity, indicating the number of units of service provided.

Some examples of financial metadata may include information for understanding the costs associated with healthcare services. This metadata may include a unique identifier for each insurance claim, known as the Claim ID, along with the total amount billed for the service (Billed Amount), the amount allowed by the insurer (Allowed Amount), and the amount paid by the insurer (Paid Amount). It may also record the member's cost-sharing responsibilities, such as co-payments, deductibles, and co-insurance, under the Member Cost Sharing category. Additionally, it may track any adjustments made to the billed amount, such as discounts or denied charges (Adjustment Amount), and may include cost center codes related to cost allocation.

Some examples of metadata associated with performance and quality metrics may include information for evaluating the delivery of healthcare services, including the Medical Loss Ratio (MLR), which represents the percentage of premiums spent on claims and healthcare quality improvement activities. Another key metric may be the Per Member Per Month (PMPM) cost, which calculates the average cost of insurance per enrolled member per eligible month. Other performance metrics may include acute admission rates, which measure the number of acute admissions per thousand members, acute readmission rates, indicating the number of patients readmitted within a specific period after discharge, and Emergency Room Visits Per Thousand (ER/k) metric, which tracks the number of ER visits per thousand members. Utilization rates, which measure the frequency of specific healthcare services, may also be part of this category.

Some examples of clinical and health outcomes metadata include information that provides insights into the health status and outcomes of members including health risk scores, which indicate the health risk level of members based on their medical history and conditions, and chronic condition indicators, which may identify the presence of chronic conditions like diabetes or hypertension. Preventive care metrics may track the receipt of preventive services, such as vaccinations and screenings, while quality measures may assess performance on various healthcare quality standards, such as HEDIS measures.

Some examples of temporal metadata may include time-related data, including the service duration which measures the length of time for services provided, an example of which may be the duration of a hospital stay. It may also encompass the time to service, which tracks the time elapsed from a member's request to the actual provision of the service, and claims processing time, which measures the time taken to process and settle a claim.

Some examples of compliance and regulatory metadata may include information related to adherence to legal and regulatory standards, including compliance indicators, which provide data on adherence to regulatory requirements, and audit trails, which may record all changes and access to the data for audit purposes. These elements may be crucial for maintaining the integrity and legality of healthcare data management.

The value estimation method 100 may advantageously evaluate thousands of permutations of the various methods described further herein. The value estimation method 100 may result in a transparent, equitable, replicable, and objective determination of various baseline and performance contractual metrics, including per member per month metrics, medical loss ratio metrics, and utilization metrics. Within the delegated model 102, a base period may be in a historical reference time frame used to evaluate the current performance of a healthcare entity. Metrics during the base period may serve as a benchmark or baseline for comparing improvements or deteriorations in performance during the subsequent performance period. In the delegated model 102, the performance period may be the current or future time frame during which the performance of the healthcare entity is measured. Improvements or declines in metrics may be assessed against the base period to determine progress or success in meeting specific goals.

Regarding the contractual metrics, the per member per month metric may be a measure of the average cost of health care utilization per enrolled member over a time period, typically one month. This metric may indicate considerations regarding budgeting and forecasting of expenses present in health insurance plans. The medical loss ratio metric may assess the percentage of premiums allocated to claims and activities that improve healthcare quality, thereby contributing to value-based care. Health insurers may be required to maintain a target medical loss ratio that meets specific regulatory standards and ensures a certain portion of premiums are spent on medical care. Utilization metrics may measure how frequently healthcare services are used. One utilization metric may be admissions per thousand (APK), which measures the number of admissions to a hospital or similar healthcare facility per thousand members in a given time period. The ER/k (Emergency Room Visits Per Thousand) metric may indicate the number of emergency room visits per thousand members. ER/k may be used to assess the accessibility and usage of primary care versus emergency care. The readmissions metric may indicate the number of patients readmitted to a hospital within a certain period after discharge, commonly 30 days. The readmissions metric may serve as an indicator of hospital quality and efficiency, with lower readmission rates generally seen as desirable.

The value estimation method 100 may advantageously provide for each entity 112 associated with the delegated model 102, particularly an APM contract associated with the delegated model 102, to analyze all potential expected values of future performance levels attributable to the delegated model 102 by using a standardized set of data and processes. This analysis of standardized data and processes disclosed herein allows for rapid analytical processing, resulting in an overall more rapid time to reach mutual agreement regarding details of the delegated model 102.

The value estimation method 100 may further include a step of transforming 126 the first set of patient-generated data 108 into a second set of patient-generated data 128. The step of transforming 126 the first set of patient-generated data 108 into the second set of patient-generated data 128 may be based upon transforming the first set of patient-generated data 108 according to a predefined delegated model parameter 130. The predefined delegated model parameter 130 may be provided as a rule or set of rules or restrictions associated with aspects of the delegated model 102, particularly with aspects of agreements associated with the delegated model 102. The step of transforming 126 and the predefined delegated model parameter 130 may truncate, censor, remove, or otherwise tailor the information of the first set of patient-generated data 108 such that the second set of patient-generated data 128 is not the same as the first set of patient-generated data 108.

For example, the predefined delegated model parameter 130 may include compensating outlier healthcare events based on cost or utilization metrics, otherwise referred to herein as cost or utilization outlier events 132. One example of compensating for the cost or utilization outlier events 132 may be truncating the first set of patient-generated data 108 for a member who costs $2M in a calendar year. In applying the predefined delegated model parameter 130, the first set of patient-generated data 108 may be truncated by setting a cost threshold at $2M, so any costs exceeding this threshold can either be capped at $2M (capped), flagged and excluded from certain analyses (row-level truncation), or result in the member excluded entirely from the analysis (member-level truncation). This example of compensating for the outlier events 132 prevents extremely high costs from skewing average cost calculations or predictive modeling outcomes. This example also normalizes cost data and mitigates the impact of rare, extremely expensive cases that are not representative of the general population.

Another example of the predefined delegated model parameter 130 may be compensating for high-cost oncology treatments 134. One exemplary way to compensate for the high-cost oncology treatments 134 is to exclude or flag high cost oncology treatments. In applying the predefined delegated model parameter 130, the first set of patient-generated data 108 may be censored by flagging the high-cost oncology treatments 134 for separate analysis or removing them from the first set of patient-generated data 108 used for general healthcare cost studies. This example of compensating for the high-cost oncology treatments 134 prevents such treatments that may be significantly more expensive that other medical treatment from distorting analysis intended to gauge typical healthcare spending. While the high-cost oncology treatments 134 is provided as an example, those of skill in the art will recognize that various treatments or costs associated with the first set of patient-generated data 108 may be flagged, censored, truncated, or otherwise separately analyzed from the first set of patient-generated data 108.

Another example of the predefined delegated model parameter 130 may be compensating for out-of-state claims 136, which may include excluding the out-of-state claims 136 from the first set of patient-generated data 108. This example predefined delegated model parameter 130 may remove from the first set of patient-generated data 108 any claims filed for services rendered out of the state. This out-of-state claims 136 example may focus subsequent processing of the second set of patient-generated data 128 on regional healthcare utilization and control for geographical variations in healthcare provision and pricing.

Another example of the predefined delegated model parameter 130 may be compensating for members with less than a certain period of eligibility during a given reconciliation period, otherwise referred to herein as short-term members 138. This example of the predefined delegated model parameter 130 may exclude data from the short-term members 138 from an annual, or other time period based, analysis of the first set of patient-generated data 108. This short-term members 138 example may improve reliability of longitudinal studies and annual comparisons of healthcare data to accurately assess healthcare utilization patterns or outcomes.

Various modifications, permutations, and variations of these examples of the predefined delegated model parameter 130 are contemplated by this disclosure. The predefined delegated model parameter 130 may be advantageously used to prepare the first set of patient-generated data 108 before subsequent analysis of the first set of patient-generated data 108, including training. The predefined delegated model parameter 130 may ensure that subsequent analysis of the first set of patient-generated data 108 is more generalizable and less likely to be influenced by non-representative data present in the first set of patient-generated data 108. Further, the step of transforming 126 the first set of patient-generated data 108 based on the predefined delegated model parameter 130 provides greater accuracy and robustness in subsequent analysis of the second set of patient-generated data 128, especially in healthcare where cost and utilization can vary dramatically due to a small number of cases.

The step of transforming 126 the first set of patient-generated data 108 into the second set of patient-generated data 128 may also be based upon data type standardization 140, feature engineering 142, member level normalization 144, or a combination thereof. Each of the data type standardization 140, the feature engineering 142, and the member level normalization 144 may be independent and distinct from the predefined delegated model parameter 130. Alternatively, the predefined delegated model parameter 130 may further include the data type standardization 140, the feature engineering 142, the member level normalization 144, or all of them.

The data type standardization 140 may transform the first set of patient-generated data 108 to ensure that certain variables or attributes identified in the first set of patient-generated data 108 are represented in a consistent format or data type. The data type standardization 140 may include converting categorial variables into numerical representations, encoding text or string variables into a standardized format, or converting date and time variables into a consistent timestamp format. Data type standardization 140 may increase the ease of performing calculations, comparisons, and analyses across different variables, increasing compatibility and consistency in downstream processing.

The feature engineering 142 may transform the first set of patient-generated data 108 to create new variables or features from the raw data to improve performance of predictive models or analytical algorithms. Feature engineering 142 may include transforming existing variables, creating interaction terms, deriving composite indices, or generating new variables based on domain knowledge or insights from the first set of patient-generated data 108. Feature engineering 142 may also involve creating standardized features that capture key characteristics of the first set of patient-generated data 108 in a format that is conducive to analysis and modeling.

The member level normalization 144 may involve standardizing and aligning the first set of patient-generated data 108 at the individual level to ensure consistency and comparability across different members or entities within the dataset. Member level normalization 144 may also standardize and align the first set of patient-generated data 108 at a group level of a determined size, the group level including a plurality of individuals. Member level normalization 144 may include scaling numerical variables to a common range, adjusting for differences in population size or demographics, or normalizing values relative to a reference population or baseline. Member level normalization 144 may mitigate biases, variations, and disparities in the data, enabling fair comparisons and assessments of individual performance or outcomes. In the first set of patient-generated data 108, member level normalization 144 may involve adjusting for differences in patient demographics, healthcare utilization patterns, or risk profiles to facilitate accurate risk adjustment, population health management, and quality measurement.

The predefined delegated model parameter 130 or any parameter used in the step of transforming 126 the first set of patient-generated data 108, including data type standardization 140, feature engineering 142, and member level normalization 144, may be variably determined by one or more of the plurality of data sources 110 associated with the delegated model 102, including the plurality of entities 112 associated with the delegated model 102. In some aspects, one entity 112 may set or determine the scope and details associated with the predefined delegated model parameter 130. In some aspects, one or more of the plurality of entities 112 may agree or otherwise negotiate or stipulate to the scope of details of the predefined delegated model parameter 130. The predefined delegated model parameter 130 may be determined individually for any one delegated model 102.

The step of transforming 126 the first set of patient-generated data 108 into the second set of patient-generated data 128 may also be defined over a predefined time period 210. The predefined time period 210 may be a user-defined time period 212, a standardized time period 214, or a dynamically determinable time period 216. The dynamically determinable time period 216 may be based on the delegated model 102, the predefined delegated model parameter 130, or the first set of patient-generated data 108. In some aspects, the predefined delegated model parameter 130 may be applied to the first set of patient-generated data 108 according to the predefined time period 210, including according to the user-defined time period 212.

The step of transforming 126 may further include a step of enhancing 218 the first set of patient-generated data 108 via feature engineering 142 by creating a derived feature 220. The step of enhancing 218 may including generating new data features or variables based on parameters derived from the first set of patient-generated data 108. The derived feature 220 may capture complex relationships or patterns within the first set of patient-generated data 108 to generate new features from the first set of patient-generated data 108. As an example, the derived feature 220 may be representative of a composite risk score based on multiple clinical risk factors or aggregating healthcare utilization metrics over the predefined time period 210.

The step of enhancing 218 may also create an interaction term 222 to capture a relationship between parameters and variables associated with the first set of patient-generated data 108. An example of the interaction term 222 may include capturing a relationship between age and comorbidities. It will be understood by those of skill in the art that various parameters, variables, and covariates of the first set of patient-generated data 108 as described herein may be evaluated in the interaction term 222.

The step of enhancing 218 may also include creating a temporal feature 224 to develop time-based features. One example of a temporal feature 224 creatable from the first set of patient-generated data 108 is the number of hospital visits over the course of the past coverage year. It will be understood by those of skill in the art that the temporal feature 224 may characterize various covariates and parameters of the first set of patient-generated data 108 according to any desired time period, including the predefined time period 210, a user-defined time period 212, or a standardized time period 214.

The step of transforming 126 may further include applying one or more standardization rules 226 to the first set of patient-generated data 108. The one or more standardization rules 226 may modify the first set of patient-generated data 108 to ensure the first set of patient-generated data 108 is represented in a consistent format or data type, including converting categorical variables into numerical representations, encoding text or string variables into a standardized format, or converting date and time variables into a consistent timestamp format. One or more standardization rules 226 may also convert disparate data types into a consistent format to facilitate subsequent processing. One or more standardization rules 226 may include categorical to numerical conversion, which may involve converting categorical variables like gender or diagnosis codes into numerical formats using techniques such as one-hot encoding or label encoding. These methods transform categorical values into binary or numerical values, making them compatible with analytical algorithms. One or more standardization rules 226 may also include text encoding, which may ensure uniformity in text representation, text or string variables are encoded into a standard format, such as ASCII or UTF-8. One or more standardization rules 226 may also include date and time normalization, which may standardize date and time variable by converting them into a uniform timestamp format, like ISO 8601. One or more standardization rules 226 may also include missing value handling, which may standardize an approach to missing or null values, including imputation, which fills in missing data, and encoding missingness indicators to clearly denote absent data for certain variables. One or more standardization rules 226 may also include scaling and transformation, which normalizes variables by scaling to a common range or distribution to standardize attributes that originally have different scales or units. Exemplary scaling and transformation techniques include min-max scaling and z-score normalization.

One or more standardization rules 226 may be implemented by providing for data ingestion, where raw data is gathered from a variety of sources such as electronic health records (EHRs), claims databases, patient surveys, and other sources present in the first set of patient-generated data 108. Next, the one or more standardization rules 226 may be established to guide the conversion of data types. This includes transforming categorical variables like gender or insurance type into numerical codes or applying one-hot encoding, standardizing date formats to a consistent timestamp, and normalizing units of measurement—for example, converting heights to meters and weights to kilograms. After establishing the one or more standardization rules 226, transformation functions may be applied to convert the raw data into standardized formats. Finally, the standardized data may undergo a validation process to check for consistency and correctness, ensuring the transformations adhere to the established standardization rules and the data is prepared for further analysis and modeling. This comprehensive approach makes diverse datasets compatible and analytically useful.

The step of transforming 126 the first set of patient-generated data 108 to the second set of patient-generated data 128 may also include member-level normalization 228, which may provide standardizing and aligning data at the individual or group level to ensure consistency and comparability across different members or entities within the second set of patient-generated data 128. Member-level normalization 228 may include scaling numerical variables to a common range, adjusting for differences in population size or demographics, or normalizing values relative to a reference population or baseline. Member-level normalization 228 may mitigate biases, variations, and disparities in the data, enabling fair comparisons and assessments of individual performance or outcomes. Member-level normalization 228 may involve adjusting for differences in patient demographics, healthcare utilization patterns, or risk profiles to facilitate accurate risk adjustment, population health management, and quality measurement.

Implementation of the member-level normalization 228 may include defining normalization criteria, such as per-member per-month (PMPM) costs. Normalization criteria may guide how data adjustments are made to ensure each member's data is comparable despite differing circumstances. Next, the first set of patient-generated data 108 may be adjusted for member-specific characteristics like age, gender, and comorbidities. These adjustments help normalize healthcare utilization metrics, making them reflective of the member's unique health context. Next, scaling techniques, such as z-score normalization, may be applied. Scaling techniques may adjust the data further to ensure that metrics are standardized across all members, facilitating fair comparisons and analyses. Finally, the member-level normalization 228 may include a validation phase, during which the data is examined to ensure that the normalization retains meaningful variability and that the adjustments enhance comparability without distorting the underlying information. This thorough approach ensures that member-level normalization effectively balances individual member differences with the need for standardized data in health analytics.

The value estimation method 100 may further include a step of checking data integrity 146 to ensure the integrity and consistency of the step of transforming 126 the first set of patient-generated data 108 into the second set of patient-generated data 128. Within the step of checking for data integrity 146 may be a step of verification 148 and a consistency check 150. The verification 148 may confirm the predefined delegated model parameter 130 has been correctly applied. The verification 148 may also confirm generally the step of transforming 126 the first set of patient-generated data 108 into the second set of patient-generated data 128 has been correctly applied. The consistency check 150 may ensure the step of transforming 126 did not introduce inconsistencies or other errors.

The value estimation method 100 may further include a step of training 152 a predictive analytics model 154. The step of training 152 the predictive analytics model 154 may include selection of appropriate machine learning algorithms 156 based on a baseline set of patient-generated data 158, the predefined delegated model parameter 130, any of the data type standardization 140, feature engineering 142, member level normalization 144, or any combination thereof. Appropriate machine learning algorithms 156 may include any one or more of the following, including combinations thereof: (1) Linear Regression: For straightforward linear relationships between features and the target variable; (2) Decision Trees: For capturing non-linear relationships and interactions between features; (3) Random Forests: An ensemble method that builds multiple decision trees and averages their predictions to improve accuracy and reduce overfitting; (4) Gradient Boosting Machines (GBM): An ensemble method that builds models sequentially, each new model correcting errors made by the previous ones, with variants including XGBoost, LightGBM, and CatBoost; (5) Neural Networks: For complex patterns and large datasets, suitable for deep learning tasks where relationships are highly non-linear.

The baseline set of patient-generated data 158 may be representatively associated with the first set of patient-generated data 108, the second set of patient-generated data 128, or both. In some aspects, the baseline set of patient-generated data 158 may include diverse samples of data that reflect distribution of key features present in the first set of patient-generated data 108 and/or in second set of patient-generated data 128, examples of such key features include age, gender, socioeconomic status, health conditions, or any other key feature that will be relevant for the step of transforming 126 by the predefined delegated model parameter 130 or relevant for subsequent analysis by the predictive analytics model 154.

In some aspects, the baseline set of patient-generated data 158 may be representative of the second set of patient-generated data 128 such that the baseline set of patient-generated data 158 has been preprocessed, by the predefined delegated model parameter 130 or by any relevant data type standardization 140, feature engineering 142, or member level normalization 144, to provide the baseline set of patient-generated data 158 in a form free from errors, duplicates, milling value, or other poor data quality characteristics that may introduce noise or bias to the training 152 of the predictive analytics model 154.

In some aspects, the baseline set of patient-generated data 158 may be representative of the second set of patient-generated data 128 such that the first set of patient-generated data 108 has been altered by feature engineering 142 so the baseline set of patient-generated data 158 include all derived features and relevant variables to ensure the predictive analytics model 154 has the necessary information to make accurate predictions.

In some aspects, the baseline set of patient-generated data 158 is representatively associated with the first set of patient-generated data 108, the second set of patient-generated data 128, or both so the baseline set of patient-generated data 158 includes similar means, variances, and ranges for numerical features and similar proportions for categorial features.

In some aspects, the baseline set of patient-generated data 158 is representatively associated with the first set of patient-generated data 108, the second set of patient-generated data 128, or both so the baseline set of patient-generated data 158 is the same as the first set of patient-generated data 108 or the second set of patient-generated data 128.

In some aspects, the baseline set of patient-generated data 158 is representatively associated with the first set of patient-generated data 108, the second set of patient-generated data 128, or both so the baseline set of patient-generated data 158 is temporally aligned to ensure the baseline set of patient-generated data 158 includes relevant historical patterns present in the first set of patient-generated data 108, the second set of patient-generated data 128, or both.

The step of training 152 the predictive analytics model 154 may further include matching the appropriate machine learning algorithms 156 to the baseline set of patient-generated data 158 to recognize and characterize relevant underlying patterns. The matching of the training 152 may include model initialization 160, including initializing the predictive analytics model 154 with defined hyperparameters 162. The defined hyperparameters 162 may include any of the following: (1) Learning Rate: Determines the step size at each iteration while moving toward a minimum of the loss function; (2) Number of Epochs: The number of times the learning algorithm will work through the entire training dataset; (3) Batch Size: The number of training samples to work through before updating the internal model parameters; (4) Number of Layers and Neurons in Neural Networks: The architecture of the network, such as the number of hidden layers and the number of neurons per layer; (5) Regularization Parameters: Such as L1 or L2 regularization, which help to prevent overfitting by penalizing large weights; (6) Kernel Type in Support Vector Machines (SVM): Determines the function used to map the input data into a higher-dimensional space; and (7) Tree Depth in Decision Trees: Limits the maximum depth of the tree to control its complexity.

The matching the appropriate machine learning algorithms 156 to the baseline set of patient-generated data 158 may also include training the baseline set of patient-generated data 158 to fit the predictive analytics model 154, where parameters of the predictive analytics model 154, including the defined hyperparameters 162, are adjusted to minimize a prediction error.

The matching the appropriate machine learning algorithms 156 to the baseline set of patient-generated data 158 may also include a cross-validation 164 to evaluate the performance and generalizability of the predictive analytics model 154. The cross-validation 164 may partition the baseline set of patient-generated data 158 into a plurality of subsets, use some of the plurality of subsets of the baseline set of patient-generated data 158 to train the predictive analytics model 154 and use the remaining of the plurality of subsets to test the predictive analytics model 154. Various types of cross-validation 164 may be used, including k-fold cross-validation, stratified k-fold cross-validation, leave-one-out cross-validation, leave-P-out cross-validation, time series cross-validation, or any combination thereof.

The step of training 152 the predictive analytics model 154 may further include model evaluation 166, where the predictive analytics model 154 is assessed for performance on the baseline set of patient-generated data 158 to ensure it generalizes well to new data, including the second set of patient-generated data 128. Model evaluation 166 may include various metrics, including the following: (1) RMSE (Root Mean Squared Error): Measures the square root of the average squared differences between predicted and actual values. Lower values indicate better performance; (2) MAE (Mean Absolute Error): Measures the average absolute differences between predicted and actual values; and (3) R² (Coefficient of Determination): Indicates the proportion of the variance in the dependent variable that is predictable from the independent variables, where values closer to 1 indicate better performance.

The step of training 152 the predictive analytics model 154 may further include hyperparameter tuning 168 to optimize the defined hyperparameters 162 of the predictive analytics model 154 to improve performance. Hyperparameter tuning 168 may include a number of methods, including the following: (1) Grid Search: Exhaustively searches through a specified subset of hyperparameters; (2) Random Search: Randomly samples hyperparameters from specified distributions; and (3) Bayesian Optimization: Uses probabilistic models to find the best hyperparameters.

The step of training 152 may further include a selection and validation 170, where the predictive analytics model 154 with the best performance is chosen and validated. The selection and validation 170 may include comparing the models of the predictive analytics model 154 by assessing the different models based on evaluation metrics, selecting the predictive analytics model 154 with the best performance on validation metrics, and performing additional validation to ensure model stability and reliability.

The predictive analytics model 154 is configured to generate, at least in part, a baseline risk rating 172, a baseline utilization rating 174, and a baseline causal inference rating 176. The baseline risk rating 172 may provide an assessment of the financial risks associated with providing healthcare services or insurance coverage to different patient populations. The baseline risk rating 172 may include the evaluation of factors such as demographic characteristics, health status, utilization patterns, and cost drivers to estimate the likelihood and severity of future healthcare expenses. The baseline risk rating 172 may include evaluating the probability and severity of healthcare expenses and claims for the covered population. The baseline risk rating 172 may be applied at various levels of applicability, including to the baseline set of patient-generated data 158 as a whole, to a subset of the baseline set of patient-generated data 158, or even on an individual data-level basis.

The baseline risk rating 172 may be based on data analysis, risk identification, risk modeling, risk factors, probability estimation, impact analysis, and scenario analysis as evaluated in the step of training 152 the predictive analytics model 154. The data analysis within the baseline risk rating 172 may analyze historical data, including claims data, demographic information, health status indicators, and utilization patterns, with some advanced analytical techniques that may be used to identify trends, patterns, and correlations within the data that may indicate potential risks or predict future healthcare costs.

The risk identification may identify and categorize different types of risks that may affect healthcare entities operating under a delegated model 102. These risks may include demographic risks (e.g., aging population), health risks (e.g., prevalence of chronic diseases), utilization risks (e.g., high-cost procedures), regulatory risks (e.g., changes in healthcare legislation), and financial risks (e.g., fluctuations in medical inflation).

Probability estimation may estimate the likelihood or probability of different outcomes or events occurring within a specified time frame. This involves calculating probabilities, confidence intervals, and risk scores based on historical data, statistical distributions, and predictive modeling techniques. Impact analysis may evaluate the potential impact or severity of identified risks on the financial performance and solvency of healthcare entities operating under a delegated model 102.

Impact analysis may include estimating the potential magnitude of losses, assessing the adequacy of reserves and capital reserves, and quantifying the potential financial implications of adverse events or scenarios.

Scenario analysis may assess the sensitivity of risk assessment outcomes to changes in key assumptions, inputs, or external factors to assist healthcare entities to understand the range of potential outcomes and develop contingency plans or risk mitigation strategies to address uncertainties and volatility in the healthcare market.

The step of training 152 the predictive analytics model 154 may also include a step of stratifying 230 the baseline set of patient-generated data 158 into a plurality of distinct risk categories 232, where the stratifying 230 is based on the predefined delegated model parameter 130, the predictive analytics model 154, or both. In one exemplary aspect, the plurality of distinct risk categories 232 may be based on demographic factors, comorbidities, and historical utilization patterns.

The step of training 152 may also include a step of assigning 234 a category risk rating 236 to each of the plurality of distinct risk categories 232. The category risk rating 236 may indicate a risk score to individual patients, groups of patients, or any subset of each of the plurality of distinct risk categories 232, where the risk score quantifies the expected future healthcare costs and/or utilization. The risk score may for example be considered as involved in data normalization by converting person-level attributes (features available from data intake that have been standardized and engineered) into an indexed numeric value of common range for all individuals fit by a model, wherein an indicated value is directly related to future risk of an adverse outcome occurring (i.e., the higher the numeric value the higher the likelihood of the bad outcome—e.g., high cost or admissions—occurring).

The step of training 152 may also include a step of generating 237 an expected cost distribution 238 for each of the plurality of distinct risk categories 232. The expected cost distribution 238 may indicate a method of cost modeling and distribution analysis, where cost modeling utilizes statistical models to estimate the expected costs for each of the plurality of distinct risk categories 232, and distribution analysis analyzes the predicted costs to understand the distribution within each of the plurality of distinct risk categories 232 (e.g., mean, median, variance).

The step of training 152 may also include receiving, via the user interface 106, a baseline risk scenario 240 reflecting one or more user-defined assumptions 242, wherein the user-defined assumptions 242 comprise healthcare costs, demographic shifts, utilization patterns, or policy changes. The baseline risk scenario 240 may be a user-generated input. These one or more user-defined assumptions 242 may reflect (1) the projected increases in healthcare costs due to inflation, changes in provider charges, or new medical technologies; changes in age distribution, population growth, or other demographic factors that affect healthcare utilization; (3) variations in the frequency and intensity of healthcare services used by patients; and (4) potential changes in health care policies, reimbursement rates, or regulations that could impact cost and utilization. The baseline risk scenario 240 may provide a baseline scenario using the one or more user-defined assumptions 242 and parameters in the predictive analytics model 154.

The step of training 152 may also include generating one or more alternative risk scenarios 244, where each of the one or more alternative risk scenarios 244 alters at least one of the one or more user-defined assumptions 242. The one or more alternative risk scenarios 244 may develop multiple alternative scenarios by adjusting the one or more user-defined assumptions 242. In an exemplary aspect, the one or more alternative risk scenarios 244 may provide (1) higher or lower health care cost inflation rates; (2) different demographic growth rates or age distributions; (3) changes in utilization patterns, such as increased chronic disease management or preventative care initiatives; and (4) potential impacts of proposed policy changes or health care reforms.

The step of training 152 may also include comparing, via the predictive analytics model 154, each of the one or more alternative risk scenarios 244 to the baseline risk scenario 240. Training 152 may also include generating a sensitivity rating 246 for each of the comparisons of each of the one or more alternative risk scenarios 244 to the baseline risk scenario 240. Comparing and generating the sensitivity rating 246 may assess the sensitivity of the predictive analytics model 154 output to changes in the one or more user-defined assumptions 242. Generating the sensitivity rating 246 may include identifying which of the one or more user-defined assumptions 242 have the most significant impact on the results, which may prioritize areas where more accurate data is needed, further research should be conducted, or patients with the greatest future variability in health outcomes are located.

The step of training 152 may also include adjusting the baseline risk rating 172 based upon the sensitivity rating 246. This adjustment may ensure robust and reliable predictive analytics models 154 are used to determine the baseline risk rating 172 and/or at least one distinct risk rating 186.

The baseline utilization rating 174 may provide an assessment of future healthcare utilization patterns and associated costs based on the claims data. The baseline utilization rating 174 within the step of training 152 the predictive analytics model 154 may include time-series analysis, regression analysis, machine learning algorithms within the appropriate machine learning algorithms 156, appropriate types of feature engineering 142, appropriate cross-validation 164, appropriate model evaluation 166 and appropriate ensemble techniques.

The baseline causal inference rating 176 may provide an evaluation of the causal impact of interventions or policy changes on healthcare outcomes and costs. The baseline causal inference rating 176 may utilize quasi-experimental designs to estimate causal relationships when randomization is not feasible, using statistical techniques to control for confounding variables and simulating the conditions of a randomized controlled trial. The baseline causal inference rating 176 within the step of training 152 the predictive analytics model 154 may include a series of matching methods 177, including propensity score matching 178 (PSM) and PSM derivatives 179, Mahalanobis distance matching 183, and exact matching methods 181.

The matching methods 177 may be based at least on the baseline set of patient-generated data 158 and the treatment subset 252 to also provide the control group 250 associated with the same treatment 254 indicated for the treatment group 248, including regarding the likelihood or probability of receiving a particular method of treatment. The matching of the treatment group 248 and control group 250 may be based on a numeric proximity of a composite set of shared attributes, as derived from the propensity score, or on an exact set of characteristics, as derived from strata in the exact matching methods 181.

The baseline causal inference rating 176 within the step of training 152 the predictive analytics model 154 may also include difference-in-differences 180 (DiD) analysis, exact matching methods 181, and instrumental variables 182 (IV). The baseline causal inference rating 176 may provide weights associated with the evaluation of the causal impact of interventions or policy changes to aid in the predictive analytics model 154 identifying relevant subsets of features to enable more accurate causal inference estimations.

Propensity score matching 178 may create comparable groups from the current and historical patient data sets by controlling for confounding variables. Within propensity score matching 178 are the following steps: (1) Identify Treatment and Control Groups: Define the treatment (e.g., a new healthcare intervention) and identify the treatment and control groups from the data sets; (2) Select Covariates: Choose relevant covariates that might influence both the treatment assignment and outcomes (e.g., age, gender, comorbidities); (3) Estimate Propensity Scores: Use logistic regression or other methods to estimate the probability (propensity score) of receiving the treatment based on the covariates; (4) Match Individuals: Match treated and control individuals based on their propensity scores using methods such as nearest neighbor matching or caliper matching; (5) Assess Balance: Evaluate the balance of covariates between matched groups to ensure comparability; and (6) Analyze Outcomes: Compare outcomes between matched groups to estimate the treatment effect.

The step of training 152 may also include defining, from the baseline set of patient-generated data 158, a treatment group 248 and a control group 250. The treatment group 248 may include portions of the baseline set of patient-generated data 158 representative of individuals who receive the intervention or treatment being studied. The control group 250 may include portions of the baseline set of patient-generated data 158 representative of individuals who do not receive the intervention or treatment being studied.

The step of training 152 may also include selecting a treatment subset 252 of the plurality of covariates, wherein the treatment subset 252 indicates of a method of treatment 254. In some aspects, matching methods 177 may match the baseline set of patient-generated data 158 based on the treatment subset 252 and may generate at least in part the treatment group 248 and the control group 250.

The step of training 152 may also include estimating a propensity score 256 of the baseline set of patient-generated data 158 based at least upon the treatment subset 252, wherein the propensity score 256 may indicate a probability of receiving the method of treatment 254. The propensity score 256 may indicate the probability of receiving the method of treatment 254 based upon the plurality of covariates. Estimating the propensity score 256 may utilize various methods including logistic regression or other appropriate methods to estimate the propensity score 256 for each individual, group, or portion selected.

The step of training 152 may also include matching 258 the treatment group 248 and the control group 250 based on various matching methods, including the propensity score 256, PSM derivatives 179, Mahalanobis distance matching 183, and exact matching methods 181. The step of matching 258 may include additional various methods, such as nearest neighbor matching, caliper matching, and stratification/interval matching. The nearest neighbor matching may provide that each treated individual is matched to one or more control individual(s) with the closest propensity score 256. The caliper matching may provide matches that are made within a specified range (caliper) of the propensity score 256. Stratification/interval matching may provide for a sample being divided into strata based on the propensity score 256, with persons being matched within each stratum.

The PSM derivatives 179 may include propensity score stratification to divide the baseline set of patient-generated data 158 into strata based on propensity scores and compare a treatment group 248 and a control group 250, as described further herein, within each stratum. Propensity score stratification may simplify matching by creating homogenous groups and comparing treatment effects within these groups. The PSM derivatives 179 may also include propensity score weighting to balance co covariates among the treatment group 248 and the control group 250. The propensity score weighting may provide a weighted estimate of the treatment effect that accounts for the distribution of propensity scores. The PSM derivatives 179 may also include propensity score regression adjustment, which may utilize propensity scores as covariates in a regression model to adjust for differences between the treatment group 248 and the control group 250. The propensity score regression adjustment may combine matching with regression to control for confounding variables.

The Mahalanobis distance matching 183 may match the treatment group 248 and the control group 250 based on a Mahalanobis distance that considers correlations between covariates. The exact matching methods 181 may match the treatment group 248 and the control group 250 based upon identified identical values for covariates. In an exemplary aspect, the exact matching methods 181 may identify or select covariates for exact matching, then match data within the treatment group 248 and the control group 250 with the same covariate values.

The matching of the treatment group 248 and the control group 250 may be based on a numeric proximity of a composite set of shared attributes between the treatment group 248 and the control group 250. The numeric proximity of the composite set of shared attributes between the treatment group 248 and the control group 250 may be derived from one or more of the matching methods 177. Including propensity score matching 178. The matching of the treatment group 248 and the control group 250 may be based on an exact set of characteristics, based on the exact matching methods 181.

Difference-in-differences 180 analysis may estimate the causal impact of interventions or policy changes by comparing pre- and post-intervention periods across matched groups. Difference-in-differences 180 analysis may include the following steps: (1) Identify Pre- and Post-Intervention Periods: Define the time periods before and after the intervention; (2) Calculate Differences: Calculate the differences in outcomes between the pre- and post-intervention periods for both treatment and control groups; (3) Compute DiD Estimator: The DiD estimator is the difference between the pre-post differences in the treatment group and the control group; and (4) Regression Approach: Use a regression model to estimate the DiD effect with controls for covariates.

Instrumental variables 182 analysis may address endogeneity issues and identify causal relationships between interventions and healthcare outcomes. Instrumental variables 182 analysis may include the following steps: (1) Identify Instrumental Variable: Find an instrument that affects the treatment assignment but not directly the outcome (e.g., policy change, geographic variation); (2) First Stage Regression: Regress the treatment on the instrumental variable and other covariates to obtain predicted values of the treatment; (3) Second Stage Regression: Regress the outcome on the predicted values of the treatment obtained from the first stage.

The value estimation method 100 may further include a step of risk assessment 184 where at least one distinct risk rating 186 is predicted by utilizing the predictive analytics model 154, the baseline risk rating 172, and the second set of patient-generated data 128. The risk assessment 184 step and prediction of at least one distinct risk rating 186 employs substantially the same methodology and tools as the determination of the baseline risk rating 172, except the prediction of at least one distinct risk rating 186 utilizes the already trained predictive analytics model 154 analyzing the second set of patient-generated data 128 and as similarly informed by the baseline risk rating 172.

The value estimation method 100 may further include a step of utilization prediction 188 where a healthcare utilization rating 190 is predicted by utilizing the predictive analytics model 154, the baseline utilization rating 174, and the second set of patient-generated data 128. The utilization prediction 188 step and prediction of the healthcare utilization rating 190 employs substantially the same methodology and tools as the determination of the baseline utilization rating 174, except the prediction of the healthcare utilization rating 190 utilizes the already trained predictive analytics model 154 analyzing the second set of patient-generated data 128 and as similarly informed by the baseline utilization rating 174.

The value estimation method 100 may further include a step of causal impact assessment 192 where a causal impact rating 194 is predicted by utilizing the predictive analytics model 154, the baseline causal inference rating 176, and the second set of patient-generated data 128. The causal impact assessment 192 and prediction of the causal impact rating 194 employs substantially the same methodology and tools as the determination of the baseline causal inference rating 176, except the prediction of the causal impact rating 194 utilizes the already trained predictive analytics model 154 analyzing the second set of patient-generated data 128 and as similarly informed by the baseline causal inference rating 176.

The value estimation method 100 may further include generating 196 a value creation estimate 198 in the delegated model 102 based upon the predefined delegated model parameter 130, the at least one distinct risk rating 186, the healthcare utilization rating 190, and the causal impact rating 194. The value creation estimate 198 may provide a monetary estimate of value creation with the delegated model 102.

In some aspects, the value creation estimate 198 may be representative of the difference between observed and expected states, which may be represented through a value function, for example in the context of a value-based care (VBC) program as follows:

$$\{ Better Care , Lower Cost \} = f ( {V}_{vbc} ( x , - x , Q , N , E , S ) ) - f ( {V}_{ffs} ( x ) ) ,$$

where “x” stands for Fee For Service (FFS) billable activity, which is what typically would be billed in a standard fee-for-service setting; “−x” represents the absence of x, essentially activities or services that are not performed, which is a crucial element in VBC as it involves understanding and monetizing what does not occur, such as the absence of utilization; “Q” stands for Quality measures that are integral to assessing the value of care delivered; “N” represents Health Related Social Needs which factor into the holistic approach of VBC, addressing broader determinants of health; “E” is for Equity, ensuring that care delivery and outcomes are fair across different populations; and “S” encapsulates Patient & Provider satisfaction, which is pivotal for a successful VBC framework as it focuses on the experiences of those giving and receiving care. The function “f(Vvbc( . . . ))” evaluates these factors in the context of a VBC program, while “f(Vffs(x))” assesses the value in a traditional fee-for-service model. The difference between these two function outputs may represent the value generated or lost under the VBC model compared to traditional models.

The value estimation method 100 may further include presenting 200 for display on a user device 202 the value creation estimate 198 in natural language text, where the natural language text indicates at least a per member per month PMPM cost adjustment 204 and a utilization adjustment 206.

The PMPM cost adjustment 204 may indicate the average cost incurred for providing healthcare services to a member of a health plan per month. PMPM cost adjustment focuses on lowering this average cost without compromising the quality of care. Some key strategies associated with PMPM cost adjustment 204 may focus on preventive care, which encourages regular screenings, vaccinations, and wellness visits. These measures are crucial for preventing chronic diseases and catching health issues early. Health promotion programs further support this effort by encouraging healthy behaviors, such as smoking cessation and weight management. For individuals with chronic conditions such as diabetes, hypertension, and Chronic Obstructive Pulmonary Disease (COPD), chronic disease management is key. This involves the development and implementation of tailored care management programs.

Care coordinators play a vital role in this strategy by ensuring that patients adhere to their treatment plans and receive timely care. Care coordination enhances the management strategy by improving the communication and collaboration between primary care providers, specialists, and other healthcare services. Tools like electronic health records (EHRs) and health information exchanges (HIEs) are instrumental in sharing patient information seamlessly, which aids in this effort. The strategy also incorporates alternative care models such as telehealth, virtual care, and remote patient monitoring (RPM), which provide convenient and cost-effective care options.

Additionally, home health programs are utilized to manage patient care in their own homes, thereby reducing the need for hospital visits. To further drive down costs, reducing hospital readmissions is critical. This can be achieved through robust post-discharge follow-up programs that ensure patients stick to their discharge plans and medication regimens. Home visits, RPM, and telehealth check-ins also help monitor patient recovery and prevent rehospitalization.

Efficient utilization of resources is another cornerstone of PMPM cost adjustment. This involves optimizing the use of diagnostic tests and procedures by adhering to clinical guidelines, which helps avoid unnecessary tests. Moreover, the implementation of value-based payment models incentivizes providers to focus on delivering high-quality and cost-effective care.

To measure the effectiveness of these strategies, PMPM costs may be calculated by dividing the total healthcare costs for a given period by the number of member-months, which is the count of members multiplied by the months they are eligible for health benefits (i.e., covered). Regular monitoring and benchmarking against industry standards or historical data, when made available, are important practices to evaluate PMPM cost adjustment. These metrics augment the analytic approach described herein to create a comprehensive framework for evaluating health improvement strategies and guide future improvements in healthcare cost management.

The utilization adjustment 206 may focus on decreasing the frequency of healthcare services used by members, particularly high-cost services like emergency room visits, hospital admissions, and specialist consultations. One strategy associated with utilization adjustment 206 may include increasing access to primary care. By enhancing the availability of primary care services, health issues can be managed early, potentially preventing them from escalating into emergencies. Practices such as implementing extended hours or offering same-day appointments in primary care settings can facilitate this approach. Another strategy may include emergency room diversion programs. These programs focus on educating members about the appropriate use of emergency services and directing them toward alternatives like urgent care centers. By developing care pathways, non-emergency cases may be redirected to lower-cost settings, thus avoiding expensive emergency room visits.

Another strategy may include care management plans for high-risk patients. By utilizing data analytics, these patients can be identified early, and targeted interventions can be provided to manage their health proactively. Case managers or health coaches may offer personalized support and follow-up, ensuring these patients receive the care they need without unnecessary hospital visits.

Another strategy may include integrating behavioral health services into primary care. This integration helps in addressing mental health issues, which are often underlying contributors to high utilization of healthcare services. Providing access to counseling and psychiatric services can prevent mental health crises that typically lead to emergency room visits.

Another strategy may include patient education and engagement. Educating patients about their conditions, treatment options, and self-care practices empowers them to manage their health more effectively. Digital tools such as patient portals, mobile apps, and remote monitoring devices can engage patients actively in their care, enhancing their ability to manage their health conditions at home.

Another strategy may include the use of data and analytics. Employing predictive analytics helps in identifying patterns and trends in healthcare utilization, which can guide clinical decision-making and tailor interventions aimed at reducing unnecessary utilization.

Metrics for evaluating utilization adjustment 206 may include the number of hospital admissions, emergency room visits, specialist referrals, and diagnostic tests per member per month. By regularly tracking and comparing these metrics over time, healthcare providers can evaluate the impact of their strategies and adjust them as needed to optimize healthcare utilization and reduce costs effectively.

The value estimation method 100 may further include a step of reconciling 208 the delegated model 102 based upon the value creation estimate 198. Reconciling 208 the delegated model 102 may include a process of compiling payments made within the delegated model 102, ensuring contractual stipulations concerning the predefined delegated model parameter 130 are met, and comparing claims submitted by one or more of the entity 112 associated with the delegated model 102 to payments made by a different entity 112 to identify any discrepancies or errors. If discrepancies or errors are found during reconciling 208, adjustments may be made within the delegated model 102 to correct any overpayments or underpayments. The step of reconciling 208 may compare the value creation estimate 198 to an observed value for the same period of time.

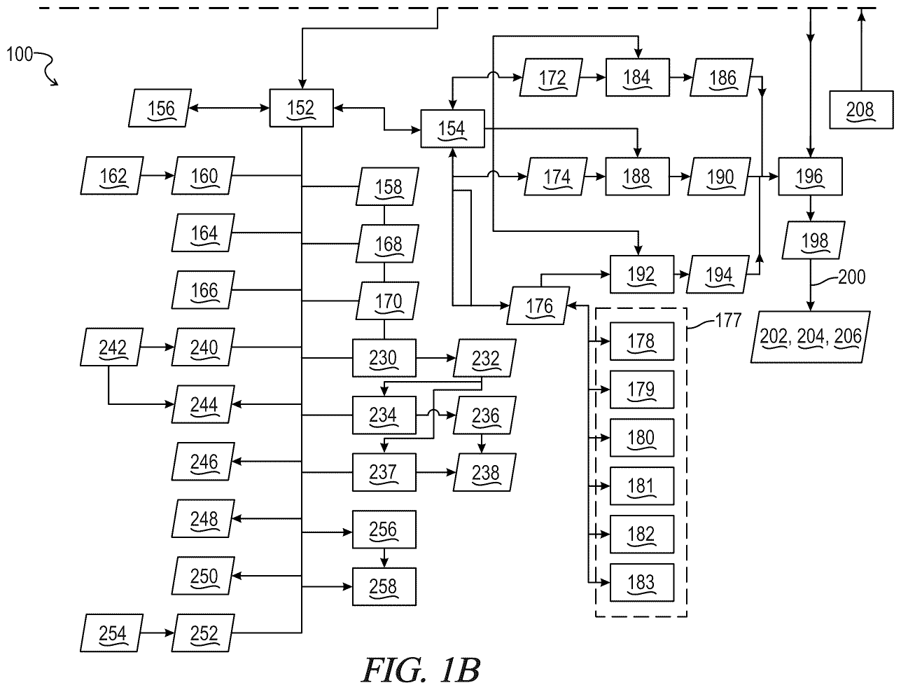

FIG. 2 illustrates an aspect of the value estimation system 300 of the present disclosure. According to one aspect of the value estimation system 300, the plurality of data sources 110 may be communicatively coupled to each other and to a server 302 via a communications network 310. The server 302 may be communicatively coupled to the communications network 310 via an input and output controller 304. The server 302 may also include a processor 306, a memory 308, and a database 312. The server 302 may utilize standard Internet protocols, such as HTTP, or secure encryption protocols, like HTTPS or other types of both secure and non-secure communication protocols as is known in the art, for communicating data, such as response data and soliciting confirmation request data from a user. The processor 306 may be used to execute software instructions for carrying out the steps disclosed with respect to the value estimation method 100.

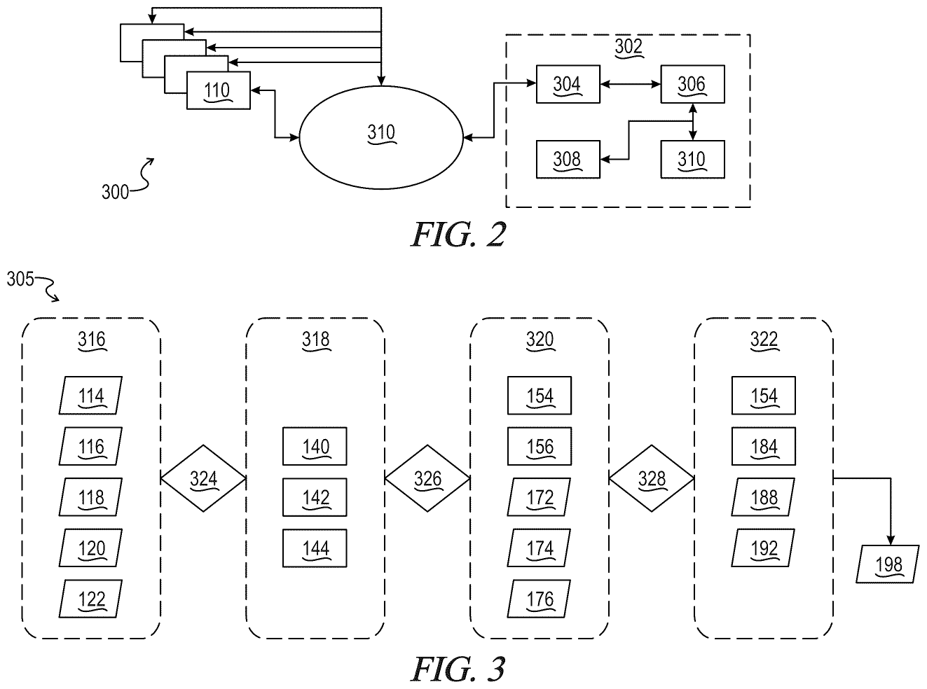

FIG. 3 illustrates an aspect of the processor 306 of the value estimation system 300. The processor 306 may include one or more modules 314 for carrying out the steps disclosed with respect to the value estimation method 100, including a data intake module 316, a database connector module 318, an expectation and simulation module 320, and a reconciliation module 322.

The data intake module 316 may receive and process the first set of patient-generated data 108, including the qualitative health assessment data 114, the medical and pharmacy claims data 116, the health benefits eligibility data 118, the demographic information 120, the geospatial data 122, and the first set of associated metadata 124.

The processor 306 may provide for a data ingestion and normalization decision 324 before the database connector module 318. The data ingestion and normalization decision 324 may provide for the transforming 126 of the first set of patient-generated data 108 from the data intake module 316. The data ingestion and normalization decision 324 may also prompt one or more of the data type standardization 140, feature engineering 142, and member level normalization 144.

The database connector module 318 may provide for preparing and transforming raw healthcare data into a format suitable for predictive modeling and actuarial risk assessment. The database connector module 318 may also be used to check for data integrity 146, verification 148, and consistency 150.

The processor 306 may provide for a data analysis decision 326 before the expectation and simulation module 320. The data analysis decision 326 may also be used to ensure data is ready for training, including checks for data integrity 146, verification 148, and consistency 150.

The expectation and simulation module 320 may receive preprocessed data, including the baseline set of patient-generated data 158 and the second set of patient-generated data 128, and may generate forecasts, simulations, and assessments using various advanced methods. The expectation and simulation module 320 may employ the use of the predictive analytics model 154 and the appropriate machine learning algorithms 156 in the training 152 to provide the baseline risk rating 172, the baseline utilization rating 174, and the baseline causal inference rating 176.

The processor 306 may provide for a reconciliation decision 328 before the reconciliation module 322. The reconciliation decision 328 may be used for validation 170 of the predictive analytics model 154.

The reconciliation module 322 may use the predictive analytics model 154, the risk assessment 184, the utilization prediction 188, and the causal impact assessment 192 to ultimately provide the value creation estimate 198. The reconciliation module 322 may also be tuned via the input and output controller 304 to account for any term relevant to the delegated model 102 and reconciliation thereof. Additional terms relevant to the delegated model 102 and reconciliation thereof may be provided by one or more of the plurality of data sources 110.

One of skill in the art will appreciate that it is theoretically possible to enumerate the full set of permutations generated by the process described herein, but such exhaustive computation may be prohibitively time consuming and computationally inefficient. Accordingly, systems and methods according to the present disclosure may contemplate employing a hybrid sampling methodology—combining statistical sampling theory with principles derived from genetic algorithms—to identify and return a representative, high-value subset of all possible permutations. By iteratively sampling candidate permutations and applying selection, crossover, and mutation operations informed by statistical criteria, the system converges on an optimal or near-optimal solution far more efficiently than brute-force enumeration. This approach enables practical deployment in real-time or resource-constrained environments without sacrificing the quality or diversity of the solution set.

The term “controller” as used herein may refer to at least general-purpose or specific-purpose processing devices, such as a central processing unit, and/or logic as may be understood by one of skill in the art, including but not limited to a microprocessor, a microcontroller, a state machine, and the like. The processor can also be implemented as a combination of computing devices, e.g., a combination of a digital signal processor (DSP) and a microprocessor, a plurality of microprocessors, one or more microprocessors in conjunction with a DSP core, or any other such configuration.

Terms such as “a,” “an,” and “the” are not intended to refer to only a singular entity, but rather include the general class of which a specific example may be used for illustration.

The phrases “in one embodiment,” “in optional embodiment(s),” and “in an exemplary embodiment,” or variations thereof, as used herein does not necessarily refer to the same embodiment, although it may.

As used herein, the phrases “one or more,” “at least one,” “at least one of,” and “one or more of,” or variations thereof, when used with a list of items, means that different combinations of one or more of the items may be used and only one of each item in the list may be needed. For example, “one or more of”' item A, item B, and item C may include, for example, without limitation, item A or item A and item B. This example also may include item A, item B, and item C, or item B and item C.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. The conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. Thus, such conditional language is not generally intended to imply that features, elements, and/or states are in any way required for one or more embodiments, whether these features, elements, and/or states are included or are to be performed in any particular embodiment.

The previous detailed description has been provided for the purposes of illustration and description. Thus, although there have been described particular embodiments of a new and useful invention, it is not intended that such references be construed as limitations upon the scope of this disclosure except as set forth in the following claims. Thus, it is seen that the apparatus of the present disclosure readily achieves the ends and advantages mentioned as well as those inherent therein. While certain preferred embodiments of the disclosure have been illustrated and described for present purposes, numerous changes in the arrangement and construction of parts and steps may be made by those skilled in the art, which changes are encompassed within the scope and spirit of the present disclosure as defined by the appended claims.

We claim:

1. A value estimation method for a delegated model, comprising:
generating a user interface configured to receive a first set of patient-generated data from one or more of a plurality of data sources associated with the delegated model, wherein the first set of patient-generated data comprises a plurality of covariates;
transforming, based at least upon a predefined delegated model parameter, the first set of patient-generated data into a second set of patient-generated data, wherein the predefined delegated model parameter is variably determined by one or more of a plurality of entities associated with the delegated model;
training a predictive analytics model by inputting into the predictive analytics model at least a baseline set of patient-generated data and the predefined delegated model parameter, where the baseline set of patient-generated data is representatively associated with the first set of patient-generated data, the second set of patient-generated data, or both, and where training the predictive analytics model is configured to generate a baseline risk rating, a baseline utilization rating, and a baseline causal inference rating;
predicting at least one distinct risk rating, based on the predictive analytics model, the baseline risk rating, the second set of patient-generated data;
predicting a healthcare utilization rating, based on the predictive analytics model, the baseline utilization rating, and the second set of patient-generated data;
predicting causal impact rating, based on the predictive analytics model, the baseline causal inference rating, and the second set of patient-generated data;
generating a value creation estimate in the delegated model based upon the predefined delegated model parameter, the at least one distinct risk rating, the healthcare utilization rating, and the causal impact rating;
presenting, by the server, for display on a user device, the value creation estimate in natural language text, wherein the natural language text indicates at least a per member per month cost adjustment and a utilization adjustment; and
reconciling the delegated model based upon the value creation estimate.

2. The value estimation method of claim 1, wherein the step of transforming comprises:
applying the predefined delegated model parameter to the first set of patient-generated data according to a user-defined time period;
enhancing the first set of patient-generated data with an engineered feature by creating a derived feature, creating an interaction term, developing a temporal feature according to the user-defined time period, or a combination thereof;
applying one or more standardization rules to the first set of patient-generated data; and
normalizing the first set of patient-generated data at a member level.

3. The value estimation method of claim 1, wherein the training comprises:
stratifying the baseline set of patient-generated data into a plurality of distinct risk categories based upon the predictive analytics model;
assigning a category risk rating to each distinct risk category of the plurality of distinct risk categories; and
generating an expected cost distribution for each distinct risk category of the plurality of distinct risk categories.

4. The value estimation method of claim 1, wherein the training comprises:
receiving, via user-generated input, a baseline risk scenario reflecting one or more user-defined assumptions, wherein the one or more user-defined assumptions comprise healthcare costs, demographic shifts, utilization patters, or policy changes;
generating one or more alternative risk scenarios, wherein each of the one or more alternative risk scenarios alters at least one of the one or more user-defined assumptions;
comparing, via the predictive analytics model, each of the one or more alternative risk scenarios to the baseline risk scenario;
generating a sensitivity rating for each of the comparisons of each of the one or more alternative risk scenarios to the baseline risk scenario; and
adjusting the baseline risk rating based upon the sensitivity rating.

5. The value estimation method of claim 1, wherein the training comprises:
defining, from the baseline set of patient-generated data, a treatment group and a control group;
selecting a treatment subset of the plurality of covariates, wherein the treatment subset indicates of a method of treatment;
estimating a matching method of the baseline set of patient-generated data based at least upon the treatment subset, wherein the control group and the treatment group comprise an equivalent probability of receiving the method of treatment; and
matching the treatment group and the control group based on the matching method.

6. The value estimation method of claim 1, wherein:
each covariate of the plurality of covariates is selected from the group consisting of: qualitative health assessment data, medical and pharmacy claims, health benefits eligibility, demographics, geospatial data, and combinations thereof.

7. A computer system for value estimation of a delegated model, comprising one or more processors configured to direct the performance of operations further comprising:
generating a user interface configured to receive a first set of patient-generated data from one or more of a plurality of data sources associated with the delegated model, wherein the first set of patient-generated data comprises a plurality of covariates;
transforming, based at least upon a predefined delegated model parameter, the first set of patient-generated data into a second set of patient-generated data, wherein the predefined delegated model parameter is variably determined by one or more of a plurality of entities associated with the delegated model;
training a predictive analytics model by inputting into the predictive analytics model at least a baseline set of patient-generated data and the predefined delegated model parameter, where the baseline set of patient-generated data is representatively associated with the first set of patient-generated data, the second set of patient-generated data, or both, and where training the predictive analytics model is configured to generate a baseline risk rating, a baseline utilization rating, and a baseline causal inference rating;
predicting at least one distinct risk rating, based on the predictive analytics model, the baseline risk rating, the second set of patient-generated data;
predicting a healthcare utilization rating, based on the predictive analytics model, the baseline utilization rating, and the second set of patient-generated data;
predicting causal impact rating, based on the predictive analytics model, the baseline causal inference rating, and the second set of patient-generated data;
generating a value creation estimate in the delegated model based upon the predefined delegated model parameter, the at least one distinct risk rating, the healthcare utilization rating, and the causal impact rating;
presenting, by the server, for display on a user device, the value creation estimate in natural language text, wherein the natural language text indicates at least a per member per month cost adjustment and a utilization adjustment; and
reconciling the delegated model based upon the value creation estimate.

8. The computer system of claim 7, wherein the one or more processors are configured to:
apply the predefined delegated model parameter to the first set of patient-generated data according to a user-defined time period;
enhance the first set of patient-generated data with an engineered feature by creating a derived feature, creating an interaction term, developing a temporal feature according to the user-defined time period, or a combination thereof;
apply one or more standardization rules to the first set of patient-generated data; and
normalize the first set of patient-generated data at a member level.

9. The computer system of claim 7, wherein the one or more processors are configured to:
stratify the baseline set of patient-generated data into a plurality of distinct risk categories based upon the predictive analytics model;
assign a category risk rating to each distinct risk category of the plurality of distinct risk categories; and
generate an expected cost distribution for each distinct risk category of the plurality of distinct risk categories.

10. The computer system of claim 7, wherein the one or more processors are configured to:
receive, via user-generated input, a baseline risk scenario reflecting one or more user-defined assumptions, wherein the one or more user-defined assumptions comprise healthcare costs, demographic shifts, utilization patters, or policy changes;
generate one or more alternative risk scenarios, wherein each of the one or more alternative risk scenarios alters at least one of the one or more user-defined assumptions;
compare, via the predictive analytics model, each of the one or more alternative risk scenarios to the baseline risk scenario;
generate a sensitivity rating for each of the comparisons of each of the one or more alternative risk scenarios to the baseline risk scenario; and
adjust the baseline risk rating based upon the sensitivity rating.

11. The computer system of claim 7, wherein the one or more processors are configured to:
define, from the baseline set of patient-generated data, a treatment group and a control group;
select a treatment subset of the plurality of covariates, wherein the treatment subset indicates of a method of treatment;
estimate a matching method of the baseline set of patient-generated data based at least upon the treatment subset, wherein the control group and the treatment group comprise an equivalent probability of receiving the method of treatment; and
match the treatment group and the control group based on the matching method.

12. The computer system of claim 7, wherein each covariate of the plurality of covariates is selected from the group consisting of: qualitative health assessment data, medical and pharmacy claims, health benefits eligibility, demographics, geospatial data, and combinations thereof.
